(19) United States
(12) Patent Application Publication
(10) Pub. No.: US 2003/0166002 A1
(43) Pub. Date: Sep. 4, 2003

(54) Triazine library with linkers

(76) Inventors: Young-Tae Chang, New York, NY (US); Ho-Sang Moon, Gyeonggi-do (KR); Sonya M. Khersonsky, New York, NY (US)

Correspondence Address: BROWDY AND NEIMARK, P.L.L.C., 624 Ninth Street, N.W., Washington, DC, 20001, US

(21) Appl. No.: 10/267,044

(22) Filed: Oct. 9, 2002

Related U.S. Application Data
(60) Provisional application No. 60/339,294, filed on Dec. 12, 2001.

(51) Int. Cl. G01N 33/53; C07D 251/18; C07D 251/48; G01N 33/543

(52) U.S. Cl. 435/7.1; 436/518; 544/204; 544/206

(57) ABSTRACT

Triazine linkers can be used as universal small molecule chips for functional proteomics and sensors. These compounds are prepared by making a first building block by adding a first amine by reductive amination of triazine, making a second building block by adding a second amine to cyanuric chloride, and combining the first and second building blocks by aminating the first building block onto one of the chloride positions of the second building block.

### CROSS-REFERENCE TO RELATED APPLICATION

The present application claims priority from non-provisional application Serial No. 60/339,294, filed Dec. 12, 2001, the entire contents of which are hereby incorporated by reference.

### FIELD OF THE INVENTION

The present invention relates to triazine linkers which can be used as universal small molecule chips for functional proteomics and sensors.

### BACKGROUND OF THE INVENTION

The Human Genome Project provided a huge amount of sequence data for dozens of thousands of genes. Elucidating the function of each gene (so-called functional genomics) is the next step in the challenge of understanding human genetics¹. Conventionally, geneticists have investigated the function of unknown genes by comparing normal phenotypes with knock-out or over-expression of the target gene, based on the assumption that the phenotypic difference is closely related to the function of the target gene. Recent developments in RNAi² and antisense techniques³ have make it possible to temporarily turn off given gene expression by targeting mRNA rather than the DNA genome itself.

A novel approach using chemical library screening to find an interesting phenotypic change by targeting specific gene products, that is, proteins, has emerged as an alternative tactic; this is called chemical genetics⁴. In chemical genetics, one chemical compound may specifically inhibit or activate one target protein (for purposes of illustration, called “protein A”). Thus, the compound is equivalent to the gene knock-out or over-expression of the corresponding gene A, as in conventional genetics.

Combinatorial library techniques⁵ facilitate the synthesis of many molecules. These techniques can be combined with high throughput screening (HTS) to screen many compounds to discover a novel, small molecule in the first step of chemical genetics study. Once one finds an intriguing small molecule, here referred to as “molecule A”, that induces a novel phenotype in cells or in an embryonic system, the next step is to identify the target protein and the biochemical pathways involved. An affinity matrix on bead or a tagged molecule (photoaffinity, chemical affinity, biotin or fluorescence) obtained by modifying molecule A, is commonly used for identifying the target protein. The target can be fished out by binding affinity of the proteins to the immobilized molecule, followed by separation on gel and sequencing by tandem mass spectrometry (MS-MS) technique. As the affinity matrix isolation usually gives multiple proteins, including non-specific binders, it is best to compare the gel results with those of control matrices side by side. Desirable control matrices will be obtained from structurally similar, molecules to molecule A which are inactive. The proteins that bind only to the active affinity matrix, without binding to the control matrices, are promising target candidates. The candidate proteins are then purified and screened in vitro with molecule A to confirm that the isolated protein is truly protein A.

As a whole, successful chemical genetics work will identify a novel gene product (i.e., protein A), and its on or off switch, small molecule pairs. By analyzing the phenotype change, the function of protein A, which is the expression product of gene A, will be discerned. At the same time, the identified small molecule key, molecule A, is a useful biochemical tool to regulate the pathway of protein A, and may be a promising drug candidate as well.

Unfortunately, the current approach of chemical genetics intrinsically contains a very difficult step, that of modifying molecule A into an affinity molecule. In order to add a linker to molecule A without adversely affecting its activity, a thorough structure-activity relationship (SAR) study of molecule A is required to find a proper site for linker addition. This site is probably a site of molecule A exposed to the solvent direction from a binding pocket in protein A. This procedure is, in many cases, extremely cumbersome, and sometimes is even completely impossible.

### SUMMARY OF INVENTION

It is an object of the present invention to overcome the aforesaid deficiencies in the prior art.

It is another object of the present invention to provide an improved method for chemical genetics.

It is a further object of the present invention to synthesize linker libraries by combinatorial methods for screening in phenotypic assays.

The present invention comprises a method for chemical genetics using library molecules carrying a linker (LL: library with linker) from the first step of the procedure. In this method, LL is synthesized by combinatorial methods and screened in phenotypic assays. The selected active compounds are directly linked to resin beads or to a tagging moiety without further SAR study using the already existing linker. Eliminating the requirement for structure-activity relationship determination dramatically accelerates the connection of function screening to the affinity matrix step. This reduces the assay time from months to days, making the chemical genetics approach much more practical and powerful than it has been heretofore.

### BRIEF DESCRIPTION OF DRAWINGS

FIG. 1 shows examples of triazine-linker compounds.

FIG. 2 shows a conventional synthetic pathway of triazine library by solution chemistry.

FIG. 3 shows an orthogonal solid phase synthesis pathway for the triazine library with linker according to the present invention.

FIG. 4 illustrates synthesis of building blocks according to the present invention.

FIG. 5 shows syntheses of triazine compound with linker.

FIG. 6 illustrates agarose bead synthesis of the triazine derivatives of the present invention.

### DETAILED DESCRIPTION OF THE INVENTION

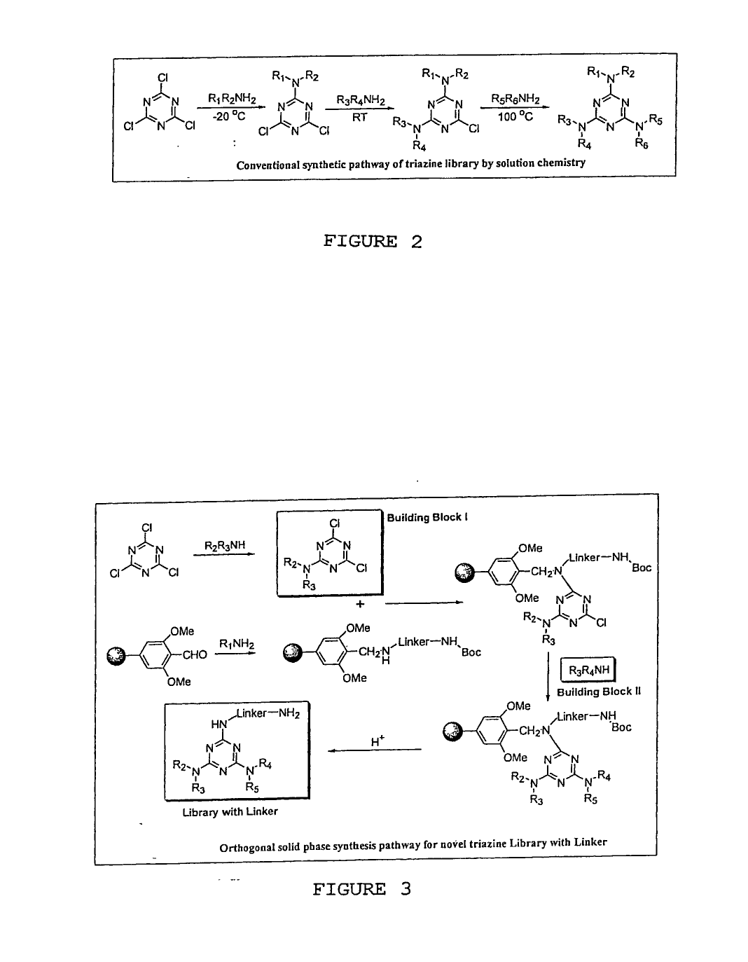

Triazine is used as the linker library scaffold. Triazines are used because they are structurally similar to purine and pyrimidine, and they have demonstrated their biological potentials in many applications. In particular, triazines have many drug-like properties, including molecular weight of less than 500, cLogP of less than 5, etc. As the triazine scaffold has three-fold symmetry, the modification is also highly flexible and able to generate diversity. Furthermore, the startng material, triazine trichloride, and all of the required building blocks, which are amines, are relatively inexpensive. Because if its ease of manipulation and the low price of the starting material, triazine has elicited much interest as an ideal scaffold for a combinatorial library, resulting in several triazine libraries having been published in the literature⁷. However, all of the reported library synthesis procedures, both for solid and solution phase chemistry, are based on sequential aminations using the reactivity differences of the three reaction sites. This is shown in FIG. 2, the conventional synthetic pathway of a triazine library by solution chemistry.

In this conventional method, the first substitution occurs at low temperatures while the second and third reactions require subsequently higher temperatures. This stepwise amination approach, however, is difficult to generalize for nucleophiles having differing reactivities. Thus, they may generate many byproducts together with the desired product.

The present invention solves the problem of byproducts using a straightforward synthetic pathway that can be used for the general preparation of a trisubstituted triazine library. The process of the present invention does not use selective amination, which requires careful monitoring of the reaction and purification steps. Instead, the present process uses three different kinds of building blocks to construct the library. The first amine (linker) is loaded onto an acid-labile aldehyde resin substrate such as by reductive amination mono- or di-methoxybenzaldehyde resins. The second amine is then added to cyanuric chloride to form a building bock with the dichlorotriazine core structure. These two building blocks are then combined by amination of the first building block onto one of the chloride positions of the second building block. Any sequential over-amination on the other chloride position is efficiently suppressed by physical segregation from any other amine available on the solid support. The third building block, which can be a primary or secondary amine, then reacts with the last chloride position to produce the trisubstituted triazine. Since all reactions are orthogonal to each other, no further purification is required after cleavage of the final compound, as shown in FIG. 3. Using this established synthetic scheme, a linker was introduced in the trisubstituted triazine library to synthesize thousands of library linker compounds in amounts of about 1-2 mg.

Syntheses of Building Blocks

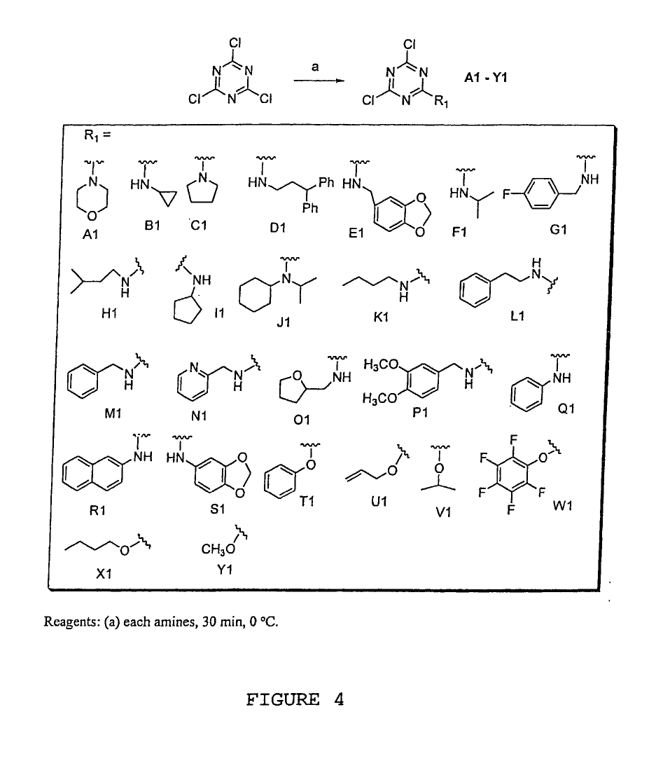

To a solution of 100 mg (0.543 mmole) cyanuric chloride, purchased from A cross Chemical Company, U.S.A., and 0.05 ml DIEA, purchased from Aldrich Chemical Company, U.S.A., in 5 ml anhydrous THF, purchased from Aldrich Chemical Company, U.S.A., was added each amine or alcohol reagent (0.652 mmol, or 1.2 eq) at 0° C. The reaction mixture was stirred for 30 minutes at 0° C. After TLC checking, the reaction mixture was filtered and the solvent removed in vacuo. The compounds were purified by column chromatography. Each compound was identified by LC-MS (Agilent 1100 model). This scheme is shown in FIG. 4, and the identification of the building blocks is shown in Table 1.

TABLE 1
Identification of Building Blocks (A1-Y1)
The products were identified LC-MS (Agilent 1100 model)

| Comp. | Mass |
|---|---|
| ID | (m + 1) |
| A1 | 235 |
| B1 | 205 |
| C1 | 219 |
| D1 | 359 |
| E1 | 299 |
| F1 | 207 |
| G1 | 273 |
| H1 | 235 |
| 11 | 233 |
| J1 | 289 |
| K1 | 221 |
| L1 | 269 |
| M1 | 255 |
| N1 | 256 |
| O1 | 249 |
| P1 | 315 |
| Q1 | 241 |
| R1 | 291 |
| S1 | 285 |
| T1 | 242 |
| U1 | 206 |
| V1 | 208 |
| W1 | 332 |
| X1 | 222 |
| Y1 | 180 |

Syntheses of Triazine Library with Linker

To a solution of 1.0 g (1.1 mmole) PAL™-aldehyde resin, purchased from Midwest Bio-Tech, U.S.A., was added 1.5 g (3.5 mmole) of Boc-linker (2-[2-amino-ethoxy-ethoxyethyl]-carbamic tert-butyl ester) in 50 ml anhydrous THF containing 10 ml of acetic acid at room temperature. The reaction mixture was stirred for one minute at room temperature and then 1.63 g (7.7.mmole, 7 eq) sodium triacetoxyborohydride was added. The reaction mixture was stirred for twelve hours and filtered. The resin was washed three times with DMF, three times with dichloromethane, three times with methanol, and three times with dichloromethane.

The next step was performed by general solid phase synthesis. To a solution of 1.0 g resin and 1 ml DIEA in 50 ml anhydrous THF at room temperature, amino-mono-substituted triazine compounds of a mono-alkoxy-substituted triazine (4 eq) was added. The reaction mixture was stirred for two hours at 60° C. and filtered. The resin was washed three times with DMF, three times with dichloromethane, three times with methanol, and three times with dichloromethane.

The final coupling step was performed by general solid phase synthesis. To the resin (10 mg) and 0.1 ml DIEA in 0.7 ml NMP was added 4 eq of each amine. The reaction mixture was stirred for two hours at 120° C. and filtered. The resin was washed three times with DMF, three times with dichloromethane, three times with methanol, and three times with dichloromethane. Resin cleavage was conducted using 10% trifluoroacetic acid in dichloromethane for 30 minutes at room temperature, after which the resin was washed with dichloromethane. The products were identified using LC-MS ((Agilent 1100 model).

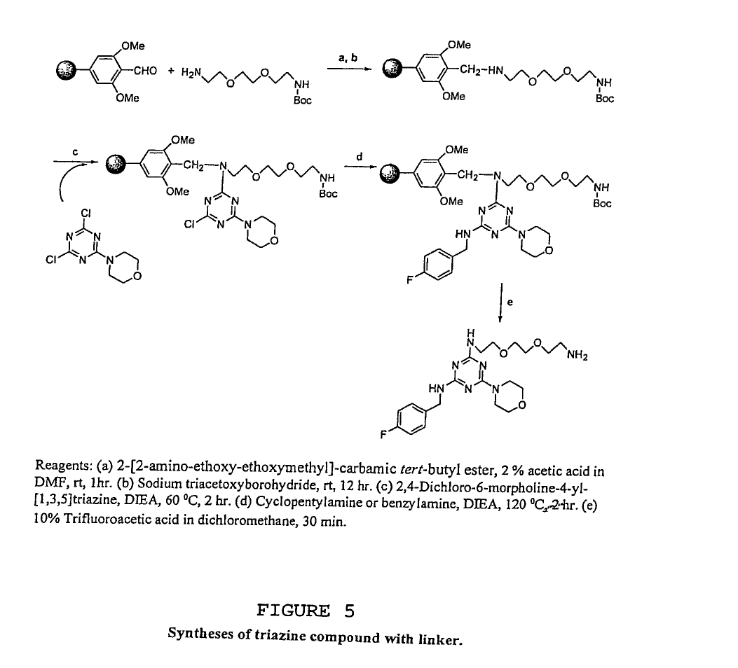

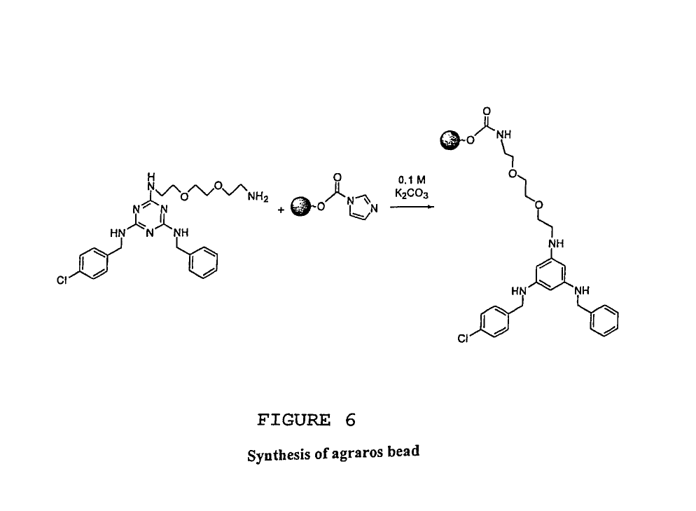

FIG. 5 illustrates syntheses of triazine compounds with linker. In this Figure, the reagents are:

a. 2-[2-amino-ethoxy-ethoxymethyl]-carbamic tert-butyl ester, 2% acetic acid in DMF, room temperature, one hour

b. sodium triacetoxyborobutyride, room temperature, for twelve hours

c. 2,4-dichloro-6-morpholine-4-yl-[1,3,5]-triazine, DIEA, at 60° C. for two hours

d. cyclopentylamine or benzylamine, DIEA,, at 120° C. for two hours

e. 10% trifluoroacetic acid in dichloromethane for 30 minutes

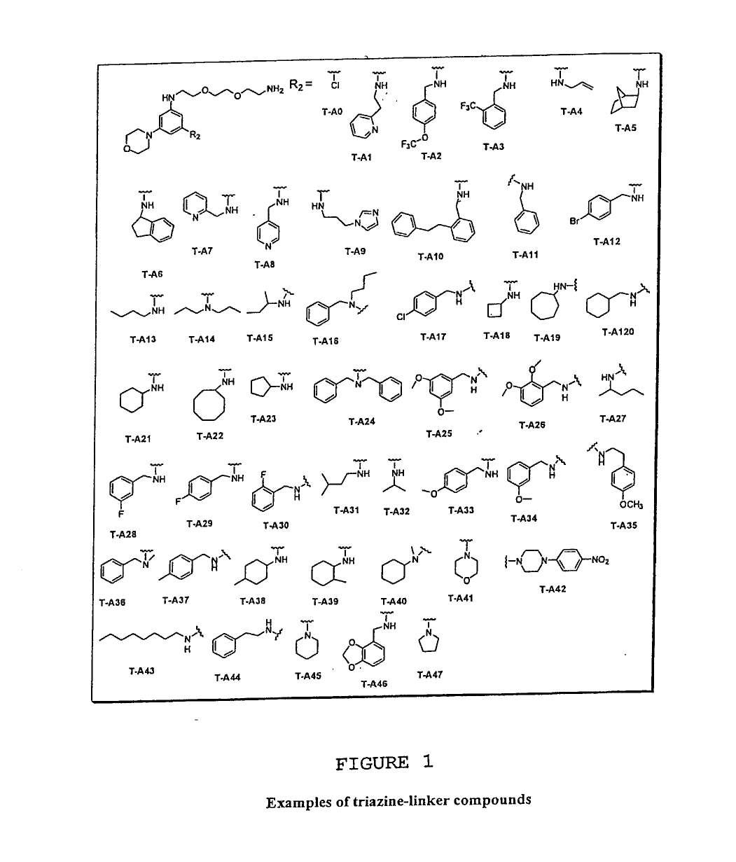

FIG. 1 illustrates examples of triazine-linker compounds. These examples are for purposes of illustration only, and are not intended to be limiting of the invention.

Table 2 illustrates compounds synthesized by the method of the present invention which were identified by LC-MS (Agilent 1100 model).

TABLE 2
Identification of Synthesized Compounds (with LC-MS).
The products were identified LC-MS (Agilent 1100 model).

| | R₁ | | | | | | | | | | | | |
|---|---|---|---|---|---|---|---|---|---|---|---|---|---|
| R₂ | A | B | C | D | E | F | G | H | I | J | K | L | M |
| 0 | 347 | 317 | 331 | 471 | 411 | 319 | 385 | 347 | 345 | 401 | 333 | 381 | 367 |
| 1 | 433 | 403 | 417 | 557 | 497 | 405 | 471 | 433 | 431 | 487 | 419 | 467 | 453 |
| 2 | 502 | 472 | 486 | 626 | 566 | 474 | 540 | 502 | 500 | 556 | 488 | 536 | 522 |
| 3 | 486 | 456 | 470 | 610 | 550 | 458 | 524 | 486 | 484 | 540 | 472 | 520 | 506 |
| 4 | 368 | 338 | 352 | 492 | 432 | 340 | 406 | 368 | 366 | 422 | 354 | 402 | 388 |
| 5 | 422 | 392 | 406 | 546 | 486 | 394 | 460 | 422 | 420 | 476 | 408 | 456 | 442 |
| 6 | 444 | 414 | 428 | 568 | 508 | 416 | 482 | 444 | 442 | 498 | 430 | 478 | 464 |
| 7 | 419 | 389 | 403 | 543 | 483 | 391 | 457 | 419 | 417 | 473 | 405 | 453 | 439 |
| 8 | 419 | 389 | 403 | 543 | 483 | 391 | 457 | 419 | 417 | 473 | 405 | 453 | 439 |
| 9 | 436 | 406 | 420 | 560 | 500 | 408 | 474 | 436 | 434 | 490 | 422 | 470 | 456 |
| 10 | 522 | 492 | 506 | 646 | 586 | 494 | 560 | 522 | 520 | 576 | 508 | 556 | 542 |
| 11 | 418 | 388 | 402 | 542 | 482 | 390 | 456 | 418 | 416 | 472 | 404 | 452 | 438 |
| 12 | 497 | 467 | 481 | 621 | 561 | 469 | 535 | 497 | 495 | 551 | 483 | 531 | 517 |
| 13 | 384 | 354 | 368 | 508 | 448 | 356 | 422 | 384 | 382 | 438 | 370 | 418 | 404 |
| 14 | 440 | 410 | 424 | 564 | 504 | 412 | 478 | 440 | 438 | 494 | 426 | 474 | 460 |
| 15 | 384 | 354 | 368 | 508 | 448 | 356 | 422 | 384 | 382 | 438 | 370 | 418 | 404 |
| 16 | 474 | 444 | 458 | 598 | 538 | 446 | 512 | 474 | 472 | 528 | 460 | 508 | 494 |
| 17 | 452 | 422 | 436 | 576 | 516 | 424 | 490 | 452 | 450 | 506 | 438 | 486 | 472 |
| 18 | 382 | 352 | 366 | 506 | 446 | 354 | 420 | 382 | 380 | 436 | 368 | 416 | 402 |
| 19 | 424 | 394 | 408 | 548 | 488 | 396 | 462 | 424 | 422 | 478 | 410 | 458 | 444 |
| 20 | 424 | 394 | 408 | 548 | 488 | 396 | 462 | 424 | 422 | 478 | 410 | 458 | 444 |
| 21 | 410 | 380 | 394 | 534 | 474 | 382 | 448 | 410 | 408 | 464 | 396 | 444 | 430 |
| 22 | 438 | 408 | 422 | 562 | 502 | 410 | 476 | 438 | 436 | 492 | 424 | 472 | 458 |
| 23 | 396 | 366 | 380 | 520 | 460 | 368 | 434 | 396 | 394 | 450 | 382 | 430 | 416 |
| 24 | 508 | 478 | 492 | 632 | 572 | 480 | 546 | 508 | 506 | 562 | 494 | 542 | 528 |
| 25 | 478 | 448 | 462 | 602 | 542 | 450 | 516 | 478 | 476 | 532 | 464 | 512 | 498 |
| 26 | 478 | 448 | 462 | 602 | 542 | 450 | 516 | 478 | 476 | 532 | 464 | 512 | 498 |
| 27 | 398 | 368 | 382 | 522 | 462 | 370 | 436 | 398 | 396 | 452 | 384 | 432 | 418 |
| 28 | 436 | 406 | 420 | 560 | 500 | 408 | 474 | 436 | 434 | 490 | 422 | 470 | 456 |
| 29 | 436 | 406 | 420 | 560 | 500 | 408 | 474 | 436 | 434 | 490 | 422 | 470 | 456 |
| 30 | 436 | 406 | 420 | 560 | 500 | 408 | 474 | 436 | 434 | 490 | 422 | 470 | 456 |
| 31 | 398 | 368 | 382 | 522 | 462 | 370 | 436 | 398 | 396 | 452 | 384 | 432 | 418 |
| 32 | 370 | 340 | 354 | 494 | 434 | 342 | 408 | 370 | 368 | 424 | 356 | 404 | 390 |
| 33 | 448 | 418 | 432 | 572 | 512 | 420 | 486 | 448 | 446 | 502 | 434 | 482 | 468 |
| 34 | 448 | 418 | 432 | 572 | 512 | 420 | 486 | 448 | 446 | 502 | 434 | 482 | 468 |
| 35 | 462 | 432 | 446 | 586 | 526 | 434 | 500 | 462 | 460 | 516 | 448 | 496 | 482 |
| 36 | 432 | 402 | 416 | 556 | 496 | 404 | 470 | 432 | 430 | 486 | 418 | 466 | 452 |
| 37 | 432 | 402 | 416 | 556 | 496 | 404 | 470 | 432 | 430 | 486 | 418 | 466 | 452 |
| 38 | 424 | 394 | 408 | 548 | 488 | 396 | 462 | 424 | 422 | 478 | 410 | 458 | 444 |
| 39 | 424 | 394 | 408 | 548 | 488 | 396 | 462 | 424 | 422 | 478 | 410 | 458 | 444 |
| 40 | 424 | 394 | 408 | 548 | 488 | 396 | 462 | 424 | 422 | 478 | 410 | 458 | 444 |
| 41 | 398 | 368 | 382 | 522 | 462 | 370 | 436 | 398 | 396 | 452 | 384 | 432 | 418 |
| 42 | 518 | 488 | 502 | 642 | 582 | 490 | 556 | 518 | 516 | 572 | 504 | 552 | 538 |
| 43 | 440 | 410 | 424 | 564 | 504 | 412 | 478 | 440 | 438 | 494 | 426 | 474 | 460 |
| 44 | 432 | 402 | 416 | 556 | 496 | 404 | 470 | 432 | 430 | 486 | 418 | 466 | 452 |
| 45 | 396 | 366 | 380 | 520 | 460 | 368 | 434 | 396 | 394 | 450 | 382 | 430 | 416 |
| 46 | 462 | 432 | 446 | 586 | 526 | 434 | 500 | 462 | 460 | 516 | 448 | 496 | 482 |
| 47 | 383 | 353 | 367 | 507 | 447 | 355 | 421 | 383 | 381 | 437 | 369 | 417 | 403 |

| | R₁ | | | | | | | | | | | |
|---|---|---|---|---|---|---|---|---|---|---|---|---|
| R₂ | N | O | P | Q | R | S | T | U | V | W | X | Y |
| 0 | 368 | 361 | 427 | 353 | 403 | 397 | 354 | 318 | 320 | 444 | 334 | 292 |
| 1 | 454 | 447 | 513 | 439 | 489 | 483 | 440 | 404 | 406 | 530 | 420 | 378 |
| 2 | 523 | 516 | 582 | 508 | 558 | 552 | 509 | 473 | 475 | 599 | 489 | 447 |
| 3 | 507 | 500 | 566 | 492 | 542 | 536 | 493 | 457 | 459 | 583 | 473 | 431 |
| 4 | 389 | 382 | 448 | 374 | 424 | 418 | 375 | 339 | 341 | 465 | 355 | 313 |
| 5 | 443 | 436 | 502 | 428 | 478 | 472 | 429 | 393 | 395 | 519 | 409 | 367 |
| 6 | 465 | 458 | 524 | 450 | 500 | 494 | 451 | 415 | 417 | 541 | 431 | 389 |
| 7 | 440 | 433 | 499 | 425 | 475 | 469 | 426 | 390 | 392 | 516 | 406 | 364 |
| 8 | 440 | 433 | 499 | 425 | 475 | 469 | 426 | 390 | 392 | 516 | 406 | 364 |
| 9 | 457 | 450 | 516 | 442 | 492 | 486 | 443 | 407 | 409 | 533 | 423 | 381 |
| 10 | 543 | 536 | 602 | 528 | 578 | 572 | 529 | 493 | 495 | 619 | 509 | 467 |
| 11 | 439 | 432 | 498 | 424 | 474 | 468 | 425 | 389 | 391 | 515 | 405 | 363 |
| 12 | 518 | 511 | 577 | 503 | 553 | 547 | 504 | 468 | 470 | 594 | 484 | 442 |
| 13 | 405 | 398 | 464 | 390 | 440 | 434 | 391 | 355 | 357 | 481 | 371 | 329 |
| 14 | 461 | 454 | 520 | 446 | 496 | 490 | 447 | 411 | 413 | 537 | 427 | 385 |
| 15 | 405 | 398 | 464 | 390 | 440 | 434 | 391 | 355 | 357 | 481 | 371 | 329 |
| 16 | 495 | 488 | 554 | 480 | 530 | 524 | 481 | 445 | 447 | 571 | 461 | 419 |
| 17 | 473 | 466 | 532 | 458 | 508 | 502 | 459 | 423 | 425 | 549 | 439 | 397 |
| 18 | 403 | 396 | 462 | 388 | 438 | 432 | 389 | 353 | 355 | 479 | 369 | 327 |
| 19 | 445 | 438 | 504 | 430 | 480 | 474 | 431 | 395 | 397 | 521 | 411 | 369 |
| 20 | 445 | 438 | 504 | 430 | 480 | 474 | 431 | 395 | 397 | 521 | 411 | 369 |
| 21 | 431 | 424 | 490 | 416 | 466 | 460 | 417 | 381 | 383 | 507 | 397 | 355 |
| 22 | 459 | 452 | 518 | 444 | 494 | 488 | 445 | 409 | 411 | 535 | 425 | 383 |
| 23 | 417 | 410 | 476 | 402 | 452 | 446 | 403 | 367 | 369 | 493 | 383 | 341 |
| 24 | 529 | 522 | 588 | 514 | 564 | 558 | 515 | 479 | 481 | 605 | 495 | 453 |
| 25 | 499 | 492 | 558 | 484 | 534 | 528 | 485 | 449 | 451 | 575 | 465 | 423 |
| 26 | 499 | 492 | 558 | 484 | 534 | 528 | 485 | 449 | 451 | 575 | 465 | 423 |
| 27 | 419 | 412 | 478 | 404 | 454 | 448 | 405 | 369 | 371 | 495 | 385 | 343 |
| 28 | 457 | 450 | 516 | 442 | 492 | 486 | 443 | 407 | 409 | 533 | 423 | 381 |
| 29 | 457 | 450 | 516 | 442 | 492 | 486 | 443 | 407 | 409 | 533 | 423 | 381 |
| 30 | 457 | 450 | 516 | 442 | 492 | 486 | 443 | 407 | 409 | 533 | 423 | 381 |
| 31 | 419 | 412 | 478 | 404 | 454 | 448 | 405 | 369 | 371 | 495 | 385 | 343 |
| 32 | 391 | 384 | 450 | 376 | 426 | 420 | 377 | 341 | 343 | 467 | 357 | 315 |
| 33 | 469 | 462 | 528 | 454 | 504 | 498 | 455 | 419 | 421 | 545 | 435 | 393 |
| 34 | 469 | 462 | 528 | 454 | 504 | 498 | 455 | 419 | 421 | 545 | 435 | 393 |
| 35 | 483 | 476 | 542 | 468 | 518 | 512 | 469 | 433 | 435 | 559 | 449 | 407 |
| 36 | 453 | 446 | 512 | 438 | 488 | 482 | 439 | 403 | 405 | 529 | 419 | 377 |
| 37 | 453 | 446 | 512 | 438 | 488 | 482 | 439 | 403 | 405 | 529 | 419 | 377 |
| 38 | 445 | 438 | 504 | 430 | 480 | 474 | 431 | 395 | 397 | 521 | 411 | 369 |
| 39 | 445 | 438 | 504 | 430 | 480 | 474 | 431 | 395 | 397 | 521 | 411 | 369 |
| 40 | 445 | 438 | 504 | 430 | 480 | 474 | 431 | 395 | 397 | 521 | 411 | 369 |
| 41 | 419 | 412 | 478 | 404 | 454 | 448 | 405 | 369 | 371 | 495 | 385 | 343 |
| 42 | 539 | 532 | 598 | 524 | 574 | 568 | 525 | 489 | 491 | 615 | 505 | 463 |
| 43 | 461 | 454 | 520 | 446 | 496 | 490 | 447 | 411 | 413 | 537 | 427 | 385 |
| 44 | 453 | 446 | 512 | 438 | 488 | 482 | 439 | 403 | 405 | 529 | 419 | 377 |
| 45 | 417 | 410 | 476 | 402 | 452 | 446 | 403 | 367 | 369 | 493 | 383 | 341 |
| 46 | 483 | 476 | 542 | 468 | 518 | 512 | 469 | 433 | 435 | 559 | 449 | 407 |
| 47 | 404 | 397 | 463 | 389 | 439 | 433 | 390 | 354 | 356 | 480 | 370 | 328 |

Table 3 illustrates structures of R₁ groups in the triazine compounds produced according to the present invention. These structures are for purposes of illustration only, and not for limitation.

TABLE 3
Structures of R₁ Group.

| R₁ | Structure |
|---|---|
| A | 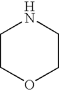 |
| B | 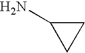 |
| C | 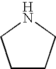 |
| D | 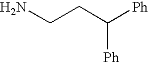 |
| E | 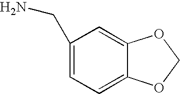 |
| F | 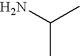 |
| G | 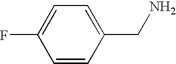 |
| H | 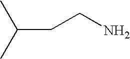 |
| I | 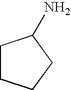 |
| J | 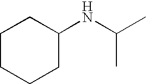 |
| K | 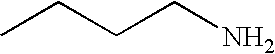 |
| L | 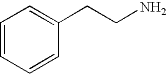 |
| M | 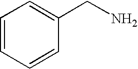 |
| N | 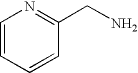 |
| O | 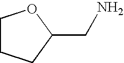 |
| P | 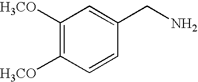 |
| Q | 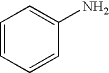 |
| R | 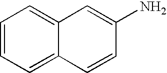 |
| S | 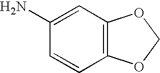 |
| T | 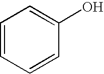 |
| U | 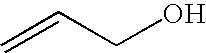 |
| V | 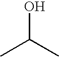 |
| W | 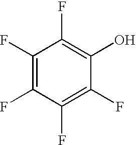 |
| X | 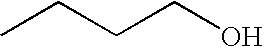 |
| Y | CH₃OH |

Structures of R₂ Group.

| R₂ | Structure |
|---|---|
| 0 | Cl |
| 1 | 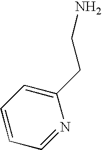 |
| 2 | 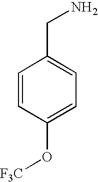 |
| 3 | 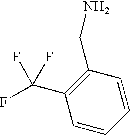 |
| 4 | 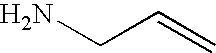 |
| 5 | 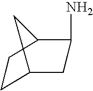 |
| 6 | 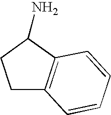 |
| 7 | 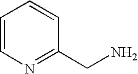 |
| 8 | 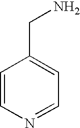 |
| 9 | 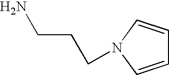 |
| 10 | 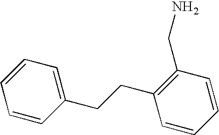 |
| 11 | 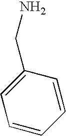 |
| 12 | 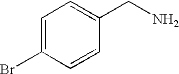 |
| 13 | 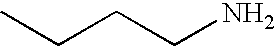 |
| 14 | 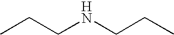 |
| 15 | 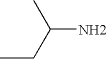 |
| 16 | 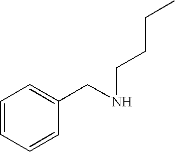 |
| 17 | 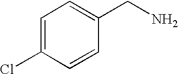 |
| 18 | 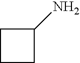 |
| 19 | 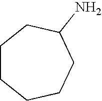 |
| 20 | 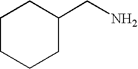 |
| 21 | 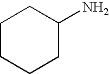 |
| 22 | 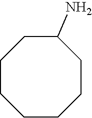 |
| 23 | 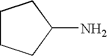 |
| 24 | 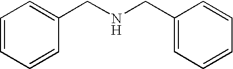 |
| 25 | 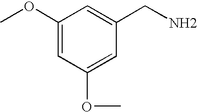 |
| 26 | 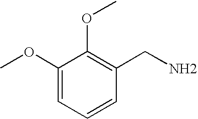 |
| 27 | 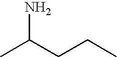 |
| 28 | 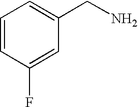 |
| 29 | 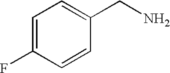 |
| 30 | 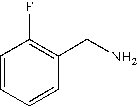 |
| 31 | 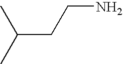 |
| 32 | 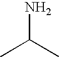 |
| 33 | 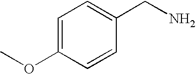 |
| 34 | 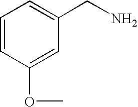 |
| 35 | 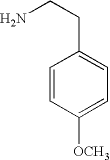 |
| 36 | 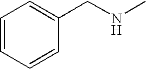 |
| 37 | 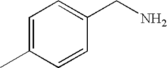 |
| 38 | 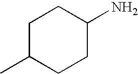 |
| 39 | 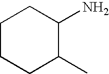 |
| 40 | 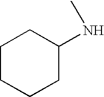 |
| 41 | 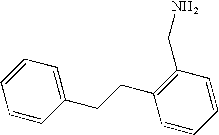 |
| 42 | 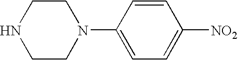 |
| 43 | 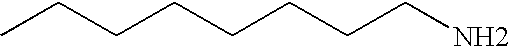 |
| 44 | 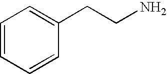 |
| 45 | 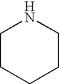 |
| 46 | 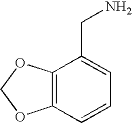 |
| 47 | 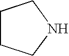 |

Generally, R₁ may be a C₁₋₁₄ alcohol or amino group, a C₁₋₁₄ alkyl group, phenyl substituted with at least one of F, Cl, methoxy, ethoxy, trifluoromethyl, or C₁₋₆ alkyl; or benzyl substituted with at least one of F, Cl, methoxy, ethoxy, trifluoromethyl, or C₁₋₆ alkyl. R₂ may be a C₁₋₁₄ amino group a C₁₋₁₄ alkyl group, phenyl substituted with at least one of F, Cl, methoxy, ethoxy, trifluoromethyl, or C₁₋₆ alkyl; or benzyl substituted with at least one of F, Cl, methoxy, ethoxy, trifluoromethyl, or C₁₋₆ alkyl.

Agarose Bead Synthesis

In a 1 ml syringe cartridge (Ppcartridge with 20 m PE frit), 1 ml of Reacti-Gel 6X in acetone (purchased from Pierce), 10 ml of crosslinked agarose, 45-165 mm, >50 mmole/ml gel was added and 2 mL ×1 0.1 M K₂CO₃ Reacti-Gel 6× in a 3 mL syringe cartridge was suspended with 1 mL of 0.1 M K₂CO₃. To this was added 100 mL (50 mM) in DMSO) triazine-linker compound with amine. The coupling buffer was removed and Tris buffer was added to block any excess reactive groups. The reaction mixture was washed twice with 10 mL H₂O and twice with 10 mL PBS.

Application of Triazine Linker Library and Affinity Matrices

The triazine linker library molecules can be used in a variety of phenotypic assays to find interesting small molecules and their binding proteins in an expeditious way. These assays include Zebrafish embryo development, morphological changes in S-pombi, membrane potential sensing in cell systems, phenotypic screening in C-elenas, muscle regeneration in newt, tumorigenesis in brain cells, apoptosis and differentiation of cancer cells, cell migration and anti-angiogenesis. The active compounds are classified depending upon their ability to induce unique morphological changes, and these are then used for affinity matrix work.

Selected linker library molecules are loaded onto activated agarose beads via their amino-end linkers as described above. These affinity matrix beads are incubated with cell or tissue extract, and found proteins run on gel. The found proteins are analyzed using MS-MS sequencing after in-gel digestion to give the peptide sequences of the target protein.

The linker library molecules can be used for making a high density small molecule chip. Thousands of linker library molecules are immobilized on a glass slide by a spotting method, which can add hundreds to thousands or molecules to a slide. The amino end of the linker is connected to an activated functional group on the slide, such as isocyanate, isothiocyanate, or acyl imidazole. Fluorescent labeled proteins with different dyes are incubated with the slide. A scanner analyzes the color to give the absolute and relative binding affinity of different proteins on each compound. For example, no color means there is no activity with any kind of proteins. A strong mixed color means that the compounds are non-specifically active with multiple proteins. Exclusively stained compounds, with a singe color, indicate a selective bind of the relevant protein. Using this technique, thousands of small molecules can be tested in a shot time using a small amount of protein. In this approach, limited numbers of purified proteins compete with each other in the presence of multiple small molecules. This approach is analogous to DNA microarray technology, which has been important in advances in functional genomics. Although there have been some reports of protein chips 8, at yet no small molecule library chip has been demonstrated. Therefore, the small molecule chips of the present invention will offer totally new techniques in the field of chemical genetics, which will expand the study of the entire genome.

Thus the present invention dramatically accelerates chemical genetics techniques by connecting phenotypic assay and affinity matrix work without any delay, rather than requiring months to year of SAR work. This powerful technique will revolutionize the study of the genome and will open a new field of chemical proteomics. Combining the binding protein data with a phenotype index will serve as a general reference of chemical knock-out. The present invention makes it possible to identify novel protein targets for drug development as well as drug candidates.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

References

1. Dhand, R. Ed., “Nature insight: Functional Genomics”, Nature, 2000, 405, 819-867.

2. (a) RNA interference: listening to the sound of silence, Zamore, P. D. Nat. Struct. Biol. 2001,8, 746-750. (b) RNAi and brain function: was McConnell on the right track?, Smalheiser, N. R.; Manev, H.; Costa, E. Trends Neurosci. 2001, 24, 216- 218. (c) Gene silencing by double-stranded RNA, Carthew, R. W. Curr. Opin. Cell Biol. 2001, 13, 244-248. (d) A conserved mechanism for post-transcriptional gene silencing?, Maine, E. M. Genome Biol. 2000, 1, 1018. (e) High-throughput reverse genetics: RNAi screens in Caenorhabditis elegans, Bargmann, C. I. Genome Biol. 2001, 2, 1005. (f) Genome-wide RNAi, Barstead, R. Curr. Opin. Chem. Biol. 2001, 5, 63-66.

3. (a) Morpholino antisense oligomers: design, preparation, and properties, Summerton, J.; Weller, D. Antisense Nucl. Acid Develop. 1997, 7, 187-195. (b) Morpholino antisense oligomers: the case for an RNase H-independent structural type, Summerton, J. Biochim. Biophys. Acta 1999, 1489, 141-158. (c) RNA-based silencing strategies in plants, Matzke, M. A.; Matzke, A. J.; Pruss, G. J.; Vance, V. B. Curr. Opin. Genet. Dev. 2001, 11, 221-227.

4. Chemical genetics resulting from a passion of synthetic organic chemistry, Schreiber, S. L. Bioorg. Med. Chem. 1998, 6, 1127-1152.

5. (a) Combinatorial Chemistry (Methods in Enzymology); Abelson, J. N. Ed.; Academic Press: New York, 1996. (b) Combinatorial Libraries-synthesis, screening and application potential; Cortese R. Ed.; Walter de Gruyter: Berlin, 1996. (c) Molecular Diversity and Combinatorial Chemistry: Libraries and Drug Discovery; Chaiken, I. M. and Janda, K. D. Ed.; American Chemical Society: New York, 1996. (d) Combinatorial Chemistry and Molecular Diversity in Drug Discovery; Gordon, E. M., Kerwin, J. F. Eds.; Wiley & Sons: New York, 1997. (e) Chemistry Synthesis and Application; Wilson, S. R., Czarnik, A. W. Eds.; Wiley & Sons: New York, 1997. (f) Combinatorial Peptide and Nonpeptide Libraried (a Handbook); Jung, G. Ed.; Wiley & Sons: New York, 1997. (g) A Practical Guide to Combinatorial Chemistry; Czarnik, A., DeWitt, S. Eds.; American Chemical Society, 1998. (h) Bunin, B. A. The Combinatorial Index; Academic Press: New York, 1998. (i) Terrett, N. K. Combinatorial Chemistry; Oxford University Press: Oxford, 1998. (j) Solid-Supported Combinatorial and Parallel Synthesis of Small-Molecular-Weight Compound Libraries; Obrecht, D., Villalgordo, J. M. Eds.; Pergamon: Netherlands, 1998. (k) Combinatorial Chemistry. Synthesis, Analysis, Screening; Jung C. Ed.; Wiley-VCH: Weinheim, 1999. (1) Dorwald, F. Z. Organic Synthesis on Solid Phase; Wiley-VCH: New York, 2000. (m) Seneci, P. Solid-Phase Synthesis and Combinatorial Technologies; Wiley & Sons: New York, 2000.

6. (a) Screening for novel antimicrobials from encoded combinatorial libraries by using a two-dimensional agar format, Silen J. L; Lu A. T.; Solas D. W.; Gore M. A.; MacLean D.; Shah N. H.; Coffin J. M.; Bhinderwala N. S.; Wang Y.; Tsutsui K. T.; Look G. C.; Campbell D. A.; Hale R. L.; Navre M.; DeLuca-Flaherty C. R. Antimicrob. Agents Chemother. 1998, 42, 1447-1453. (b) A strategy for the generation of biomimetic ligands for affinity chromatography. Combinatorial synthesis and biological evaluation of an IgG binding ligand, Teng, S. F.; Sproule, K.; Hussain, A.; Lowe, C. R. J. Mol. Recognit.1999, 12, 67-75. (c) Design, synthesis and evaluation of biomimetic affinity ligands for elastases, Filippusson, H.; Erlendsson, L. S.; Lowe, C. R. J. Mol. Recognit. 2000, 13, 370-381.

7. (a) Incorporation of carbohydrates and peptides into large triazine-based screening libraries using automated parallel synthesis, Gustafson, G. R.; Baldino, C. M.; O'Donnel, M. E.; Sheldon, A. Tarsa, R. J.; Verni, C. J.; Coffen, D. L. Tetrahedron 1998, 54, 4051-4065. (b) Spatially Addressed Synthesis of Amino- and Amino-oxy-Substituted 1,3,5-Triazine Arrays on Polymeric Membranes, Scharn, D.; Wenschuh, H.; Reineke, U.; Schneider-Mergener, J.; Germeroth, L. J. Comb. Chem. 2000, 2, 361-369. (c) Solution- and solid-phase synthesis of combinatorial libraries of trisubstituted 1,3,5-triazines, Masquelin, T.; Meunier, N.; Gerber, F.; Rosse, G. Heterocycles 1998, 48, 2489-2505. (d) Libraries of N-alkylamino heterocycles from nucleophilic aromatic substitution with purification by solid supported liquid extraction, Johnson, C. R.; Zhang, B.; Fantauzzi, P.; Hocker, M.; Yager, K. M. Tetrahedron 1998, 54, 4097-4106. (e) Library generation through successive substitution of trichlorotriazine, Stankova, M.; Lebl, M. Mol. Diversity 1996, 2, 75-80.

8. Printing proteins as microarrays for high-throughput function determination, MacBeath, G.; Schreiber, S. L. Science 2000, 289, 1760-1763.

9. ArQule: Incorporation of Carbohydrates and Peptides into Large Triazine-Based Screening Libraries Using Automated Parallel Synthesis. Gustafson, G. R.; Baldino, C. M.; O'Donnel, M. E.; Sheldon, A. Tarsa, R. J.; Verni, C. J.; Coffen, D. L. Tetrahedron 1998, 54, 4051-4065.

10. Selectide Corporation: Library generation through successive substitution of trichlorotriazine. Stankova, M.; Lebl, M. Mol. Diversity 1996, 2, 75-80.

11. Arris Pharmaceutical: Johnson, Charles R.; Zhang, Birong; Fantauzzi, Pascal; Hocker, Michael; Yager, Kraig M. Libraries of N-alkylamino heterocycles from nucleophilic aromatic substitution with purification by solid supported liquid extraction. Tetrahedron (1998), 54(16), 4097-4106. CODEN: TETRAB ISSN:0040-4020. CAN 128:308415 AN 1998:233899

12. Abbott: Hajduk, Philip J.; Dinges, Juergen; Schkeryantz, Jeffrey M.; Janowick, David; Kaminski, Michele; Tufano, Michael; Augeri, David J.; Petros, Andrew; Nienaber, Vicki; Zhong, Ping; Hammond, Rachel; Coen, Michael; Beutel, Bruce; Katz, Leonard; Fesik, Stephen W. Novel Inhibitors of Erm Methyltransferases from NMR and Parallel Synthesis. J. Med. Chem. (1999), 42(19), 3852-3859. CODEN: JMCMAR ISSN:0022-2623. CAN 131:308768 AN 1999:567005

13. Humboldt-Universitaet: Scharn, Dirk; Wenschuh, Holger; Reineke, Ulrich; Schneider-Mergener, Jens; Germeroth, Lothar. Spatially Addressed Synthesis of Amino- and Amino-Oxy-Substituted 1,3,5-Triazine Arrays on Polymeric Membranes. J. Comb. Chem. (2000), 2(4), 361-369. CODEN: JCCHFF ISSN:1520-4766. CAN 133:135605 AN 2000:355907

14. Hoffmann-La Roche: Masquelin, Thierry; Meunier, Nathalie; Gerber, Fernand; Rosse, Gerard. Solution- and solid-phase synthesis of combinatorial libraries of trisubstituted 1,3,5-triazines. Heterocycles (1998), 48(12), 2489-2505. CODEN: HTCYAM ISSN:0385-5414. CAN 130:196625 AN 1999:50090

15. Affymax: Silen J L; Lu A T; Solas D W; Gore M A; MacLean D; Shah N H; Coffin J M; Bhinderwala N S; Wang Y; Tsutsui K T; Look G C; Campbell D A; Hale R L; Navre M; DeLuca-Flaherty C R Screening for novel antimicrobials from encoded combinatorial libraries by using a two-dimensional agar format. ANTIMICROBIAL AGENTS AND CHEMOTHERAPY (1998 Jun), 42(6), 1447-53. Journal code: 6HK. ISSN:0066-4804. DN 98287588 PubMed ID 9624492 AN 1998287588

16. U. Cambridge: Teng, Su Fern; Sproule, Kenny; Hussain, Abid; Lowe, Christopher R. A strategy for the generation of biomimetic ligands for affinity chromatography. Combinatorial synthesis and biological evaluation of an IgG binding ligand. J. Mol. Recognit. (1999), 12(1), 67-75. CODEN: JMORE4 ISSN:0952-3499. CAN 131:30755 AN 1999:224014

17. U. Iceland: Filippusson, Horour; Erlendsson, Lyour S.; Lowe, Christopher R.. Design, synthesis and evaluation of biomimetic affinity ligands for elastases. J. Mol. Recognit. (2000), 13(6), 370-381, 3 Plates. CODEN: JMORE4 ISSN:0952-3499. CAN 134:174701 AN 2000:857136

18. Closest in terms of biological activity and structure: Abbott 1: Henkin, Jack; Davidson, Donald J.; Sheppard, George S.; Woods, Keith W.; McCroskey, Richard W. Preparation of triazine-2,4-diamines as angiogenesis inhibitors. PCT Int. Appl. (1999), 66 pp. CODEN: PIXXD2 WO 9931088 Al 19990624 CAN 131:58855 AN 1999:404953

19. Abbott 2: Shock, Richard U. 2-[2-(5-Nitrofuryl)]-4,6-diamino-s-triazine. US 2885400 19590505 CAN 53:94898 AN 1959:94898

20. ArQule: Coffen, David L.; Hogan, Joseph C., Jr. Synthesis and use of biased arrays. PCT Int. Appl. (1998), 53 pp. CODEN: PIXXD2 WO 9846551 Al 19981022 CAN 129:302216 AN 1998:706192

21. Trustees of Boston University: Panek, James S.; Zhu, Bin. Synthesis of aromatic compounds by Diels-Alder reaction on solid support. PCT Int. Appl. (1998), 26 pp. CODEN: PIXXD2 WO 9816508 A2 19980423 CAN 128:308494 AN 1998:251157

22. ISIS Pharmaceuticals, Inc.: Cook, P. Dan; An, Haoyun. Preparation of compounds or combinatorial libraries of compounds having a plurality of nitrogenous substituents. PCT Int. Appl. (1998), 187 pp. CODEN: PIXXD2 WO 9805961 Al 19980212 CAN 128:180338 AN 1998:112497

23. Hoffman-La Roche: Huber, Ulrich. Silanyl-triazines as light screening compositions. Eur. Pat. Appl. (1999), 26 pp. CODEN: EPXXDW EP 933376 A2 19990804 CAN 131:130123 AN 1999:505797

What is claimed is:

1. A trisubstituted triazine library.

2. A method for preparing a trisubstituted triazine library comprising:
a. making a first building block by adding a first amine by reductive amination of triazine;
b. making a second building block by adding a second amine to cyanuric chloride;
c. combining said first building block with said second building block by aminating the first building block onto one of the chloride positions of the second building block;
d. reacting a third building block with the combined first and second building blocks to produce a trisubstituted triazine.

3. The process according to claim 2 wherein the first amine is selected from the group consisting of amines substituted with at least one of a C₁₋₁₄ alcohol or amino group, a C₁₋₁₄ alkyl group, phenyl substituted with at least one of F, Cl, methoxy, ethoxy, trifluoromethyl, and C₁₋₆ alkyl; and benzyl substituted with at least one of F, Cl, methoxy, ethoxy, trifluoromethyl, and C₁₋₆ alkyl; and the second amine is substituted with at least one of a C₁₋₁₄ amino group , a C₁₋₁₄ alkyl group, phenyl substituted with at least one of F, Cl, methoxy, ethoxy, trifluoromethyl, and C₁-₆ alkyl; and benzyl substituted with at least one of F, Cl, methoxy, ethoxy, trifluoromethyl, and C₁₋₆ alkyl.

4. The process according to claim 2 wherein the first building block is selected from compounds of the formula:
wherein R₁ is selected from the group consisting of

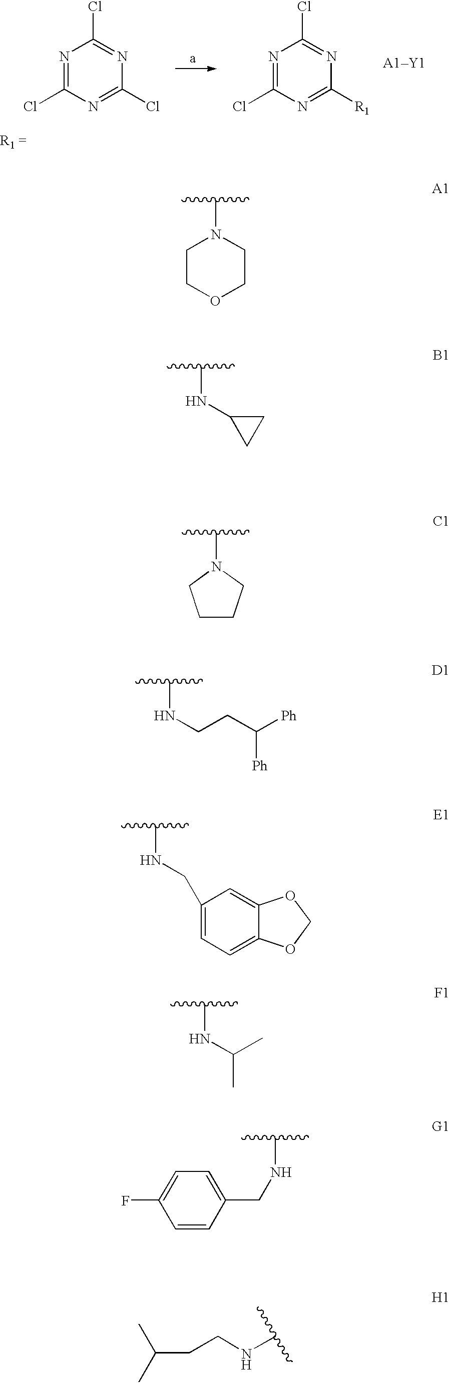

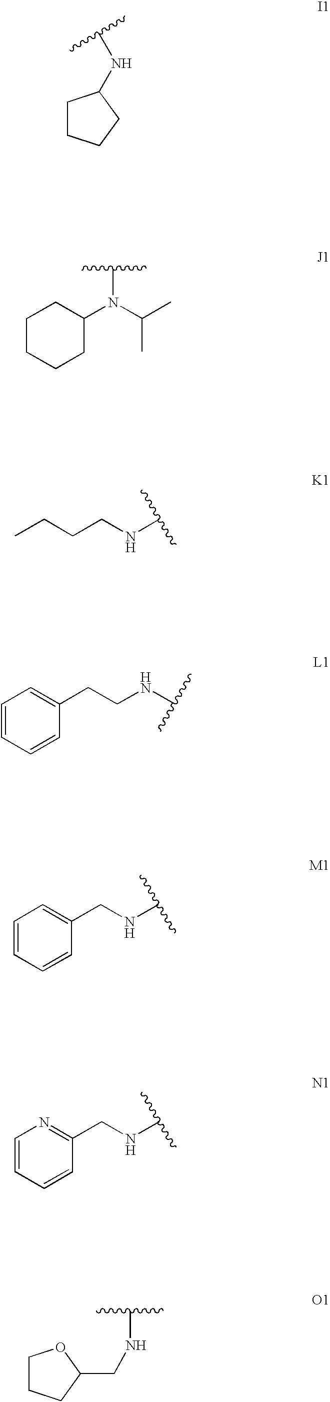

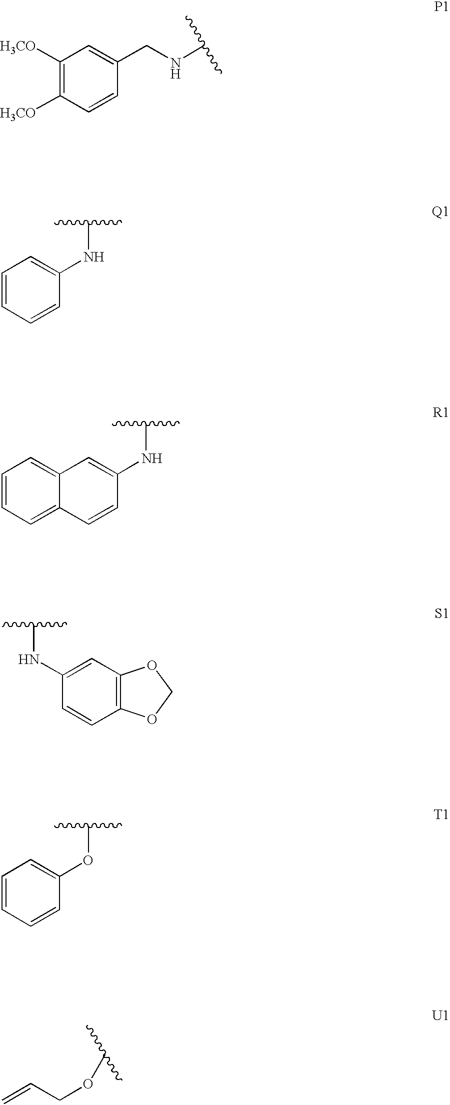

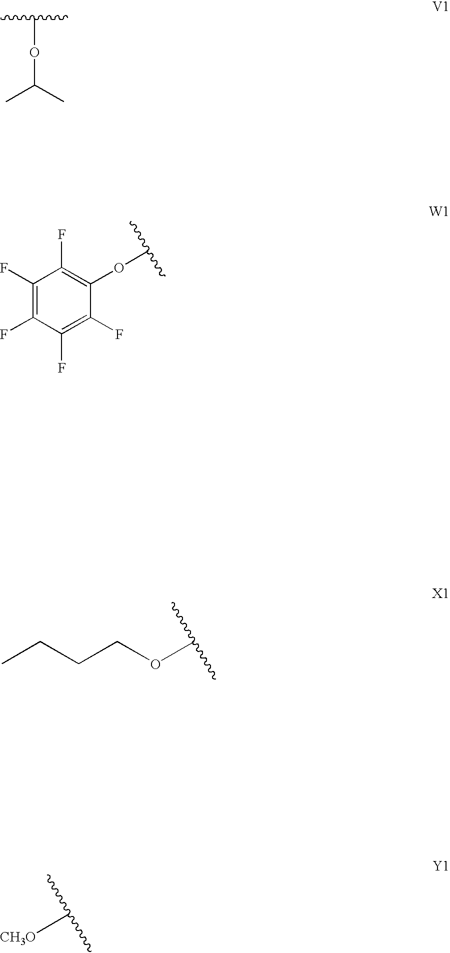

5. A process for synthesizing a triazine library with linker comprising reacting a trisubstituted triazine according to claim 1 with a linker.

6. The process according to claim 5 wherein the linker is 2-[2-amino-ethoxy-ethyoxyethyl]carbamic tert-butyl ester.

7. Triazine-linker compounds comprising a trisubstituted triazine bonded to a linker.

8. The compounds according to claim 7 selected from compounds of the following formula: 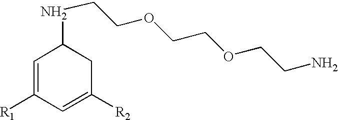
wherein R₁ is selected from the group consisting of 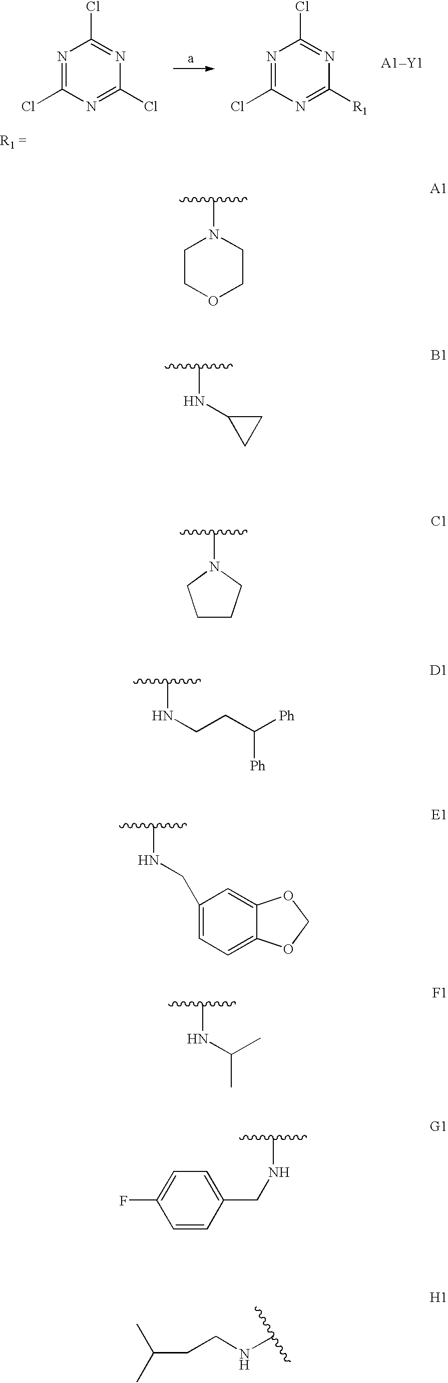 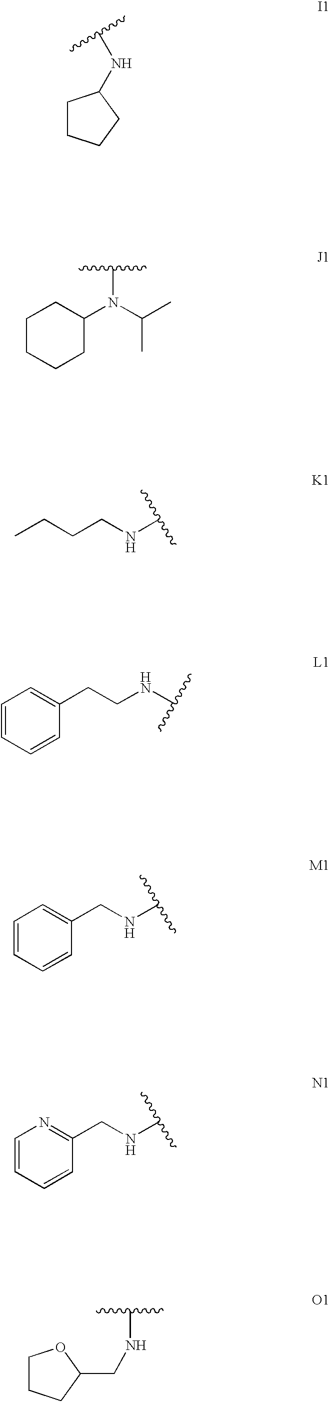 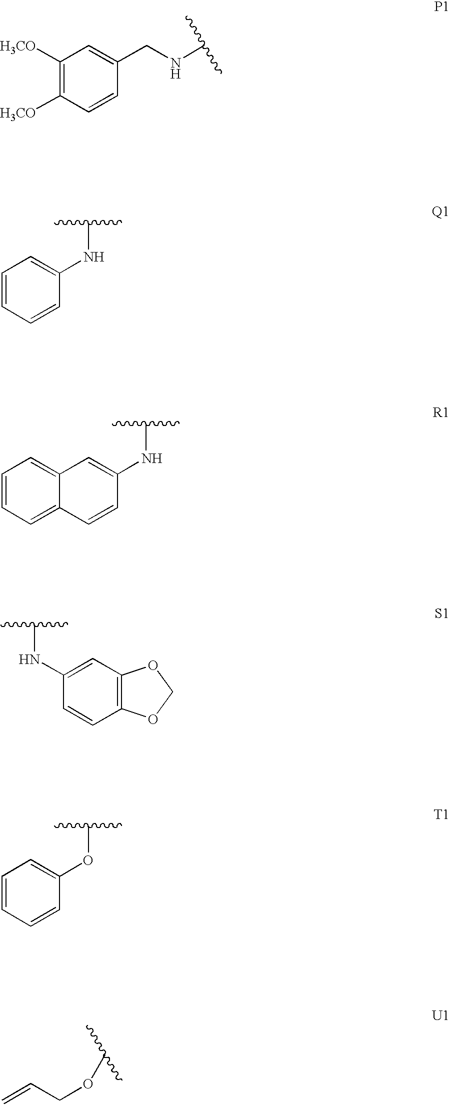 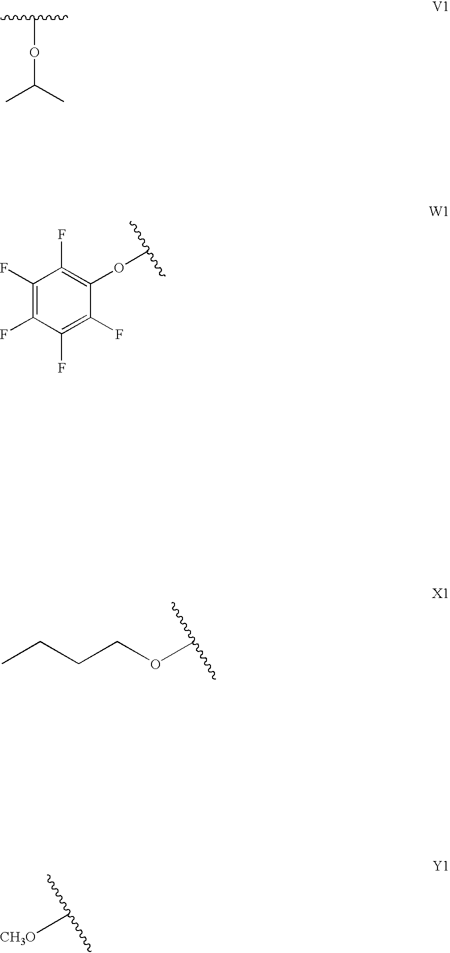
wherein R₂ is selected from the group consisting of

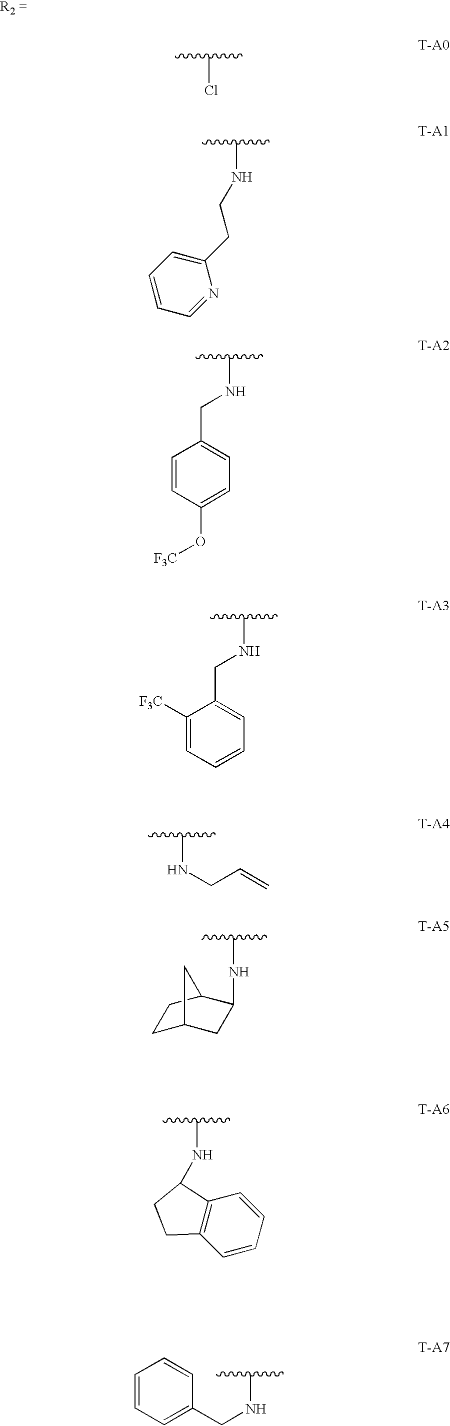

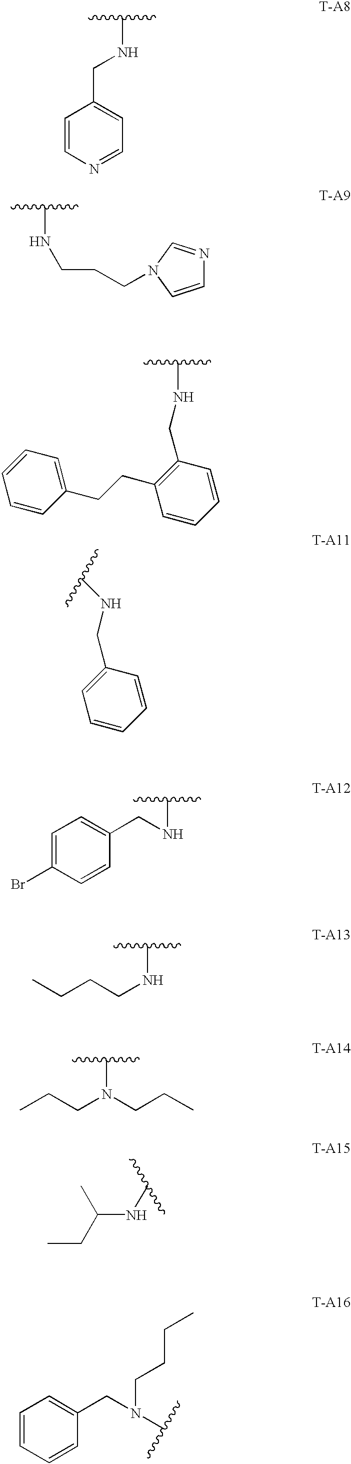

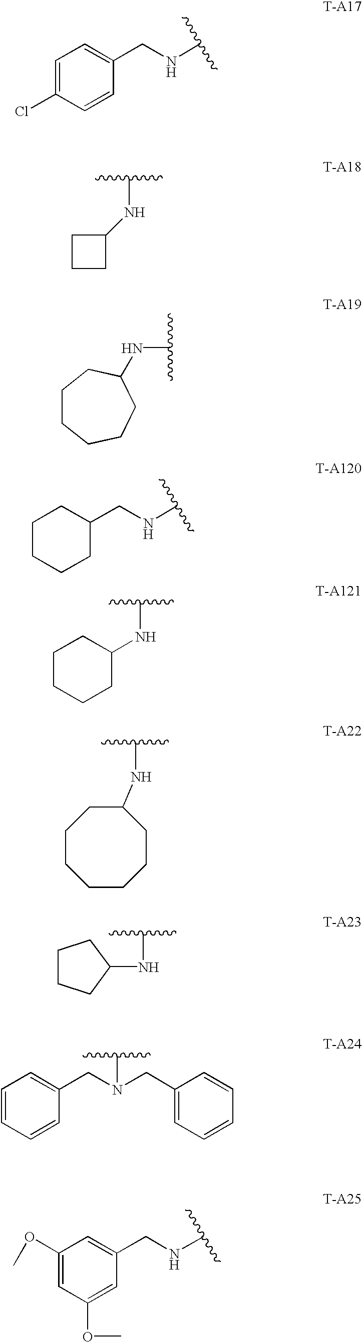

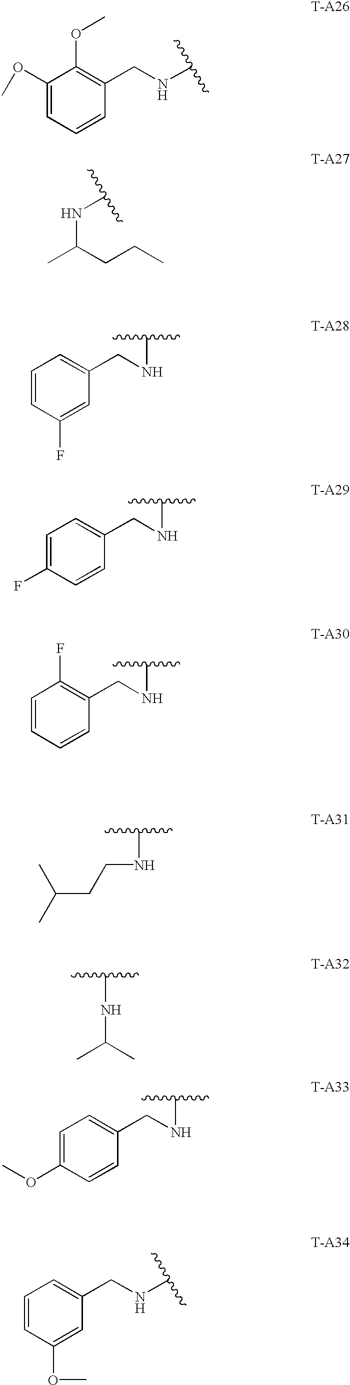

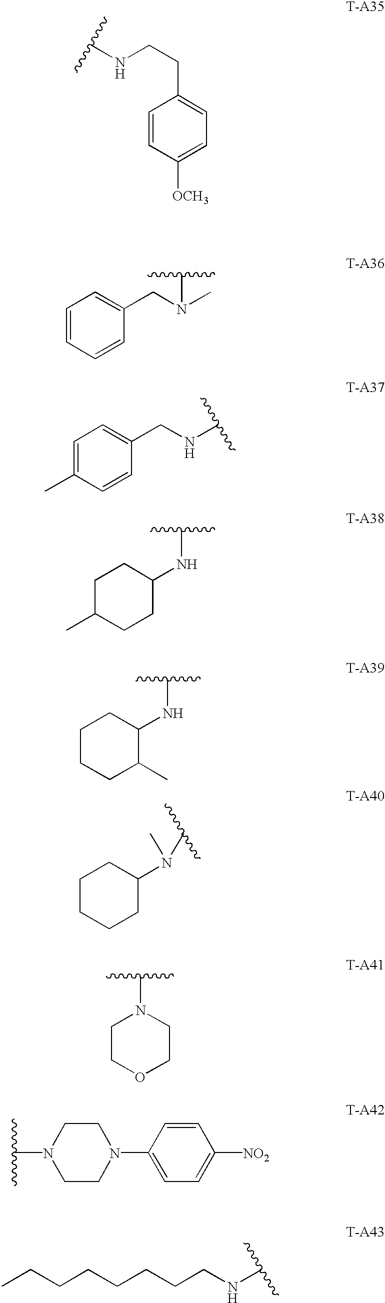

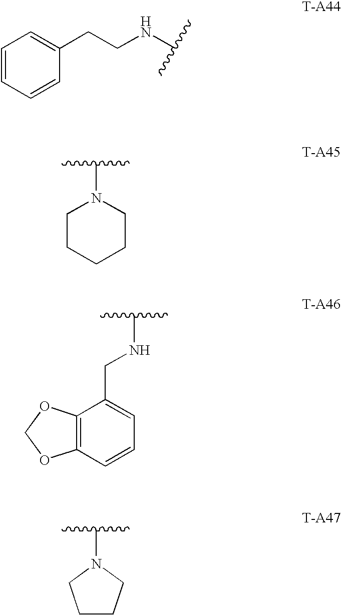

9. Affinity matrix beads comprising a triazine linker compound according to claim 7 loaded onto activated beads.

10. The affinity matrix beads according to claim 9 wherein the beads are agarose.

11. A high density small molecule chip comprising a surface onto which are linked triazine linker compounds according to claim 7.

12. The high density small molecule chip according to claim 11 wherein the surface is a glass slide.

13. The high density small molecule chip according to claim 11 wherein the amino end of the linker is connected to an activated functional group on the surface.

14. The high density small molecule chip according to claim 13 wherein the activated functional group is selected from the group consisting of isocyanate, isothiocyanate, and acyl imidazole.

15. A method for determining the binding affinity of proteins to a plurality of molecules comprising incubating a high density small molecule chip according to claim 11 with a plurality of labeled proteins, and analyzing the labels to determine which molecule have affinity for which proteins.

16. The method according to claim 15 wherein the label is a florescent label.
